(19) United States
(12) Patent Application Publication
(10) Pub. No.: US 2025/0388719 A1
(43) Pub. Date: Dec. 25, 2025

(54) NOVEL CURING AGENT HAVING ESTER AND AMIDE GROUPS, METHOD FOR PREPARING THE SAME, EPOXY COMPOSITION, CURED PRODUCT, AND ARTICLE COMPRISING THE SAME

(71) Applicant: KOREA INSTITUTE OF INDUSTRIAL TECHNOLOGY, Cheonan-si (KR)

(72) Inventors: Hyun Aee CHUN, Suwon-si (KR); Sook Yeon PARK, Gunpo-si (KR); Yun Ju KIM, Seoul (KR); Su Jin PARK, Osan-si (KR)

(21) Appl. No.: 18/960,690

(22) Filed: Nov. 26, 2024

(30) Foreign Application Priority Data
Jun. 24, 2024 (KR) 10-2024-0082082

(51) Int. Cl. C08G 59/46; C07C 231/02; C07F 7/18; C08G 59/62; C08K 3/36; C08L 63/00

(52) U.S. Cl. CPC C08G 59/46; C07C 231/02; C07F 7/1892; C08G 59/621; C08K 3/36; C08L 63/00

(57) ABSTRACT

The present disclosure relates to a novel curing agent having ester and amide groups simultaneously attached to a benzene unit exhibiting excellent low dielectric loss characteristics (low dissipation factor or low Df) and processability, a method for preparing the same, an epoxy composition, a cured product, and an article comprising the same. Excellent low dielectric loss and processability are achieved by the epoxy composition comprising the novel curing agent of the present disclosure and epoxy resin. Therefore, the epoxy composition comprising the novel curing agent of the present disclosure is suitable for use as an insulating film, an epoxy film for semiconductor packaging, an adhesive film, a build-up film, a substrate film, an epoxy molding material, or the like due to these improved properties.

### CROSS-REFERENCE TO RELATED APPLICATION

This application claims benefit of priority to Korean Patent Application No. 10-2024-0082082 filed on Jun. 24, 2024, in the Korean Intellectual Property Office, the disclosure of which is incorporated herein by reference in its entirety.

### BACKGROUND

### 1. Field

The present disclosure relates to a novel curing agent having ester and amide groups, a method for preparing the same, an epoxy composition, a cured product, and an article comprising the same. Specifically, the present disclosure relates to a novel curing agent simultaneously having ester and amide groups in a benzene unit exhibiting excellent low dielectric loss characteristics (low dissipation factor or low Df) and processability, a method for preparing the same, an epoxy composition, a cured product, and an article comprising the same.

### 2. Description of Related Art

With an advancement of semiconductor packaging technology, it is required to transmit large amounts of data as quickly as possible and without transmission loss. Using a high dielectric loss of the epoxy material as a semiconductor packaging material results in the significant data transmission loss and deteriorates the quality of the transmission signal, and thus, the low dielectric loss (low dielectric dissipation factor or low Df) characteristic of semiconductor insulating material is a significantly critical characteristic for high-quality high-speed data transmissions.

As disclosed in Korean Patent No. 2051374, the most representative method for lowering dielectric loss of an epoxy material is to use an active ester compound as a curing agent. The dielectric loss characteristics of an epoxy insulating material have been greatly improved by the use of a conventional active ester as curing agent, but there is a problem in that processability, especially the desmear properties of an epoxy film is diminished.

Accordingly, in order to manufacture semiconductor components for ultra-high-speed semiconductor data transmission, the development of an epoxy insulating material that may simultaneously satisfy excellent dielectric loss (Df) (i.e., low Df) characteristics and processability of the epoxy insulating material is required.

### PRIOR ART REFERENCE

### Patent Document

(Patent Document 1) Korean Patent No. 2051374

### SUMMARY

As described above, the development of a material that satisfies both excellent low dielectric loss (Df) characteristics and processability, specifically desmearability, in an epoxy cured product is required. The inventors of the present disclosure have discovered that a novel curing agent simultaneously having ester and amide groups in a benzene unit of the present disclosure not only exhibits improved processability in an epoxy material, specifically, desmearability, but also satisfies excellent low dielectric loss (Df) characteristics in an epoxy cured product, and the present disclosure is based thereon. Accordingly, the present disclosure provides a novel curing agent having ester and amide groups simultaneously in a benzene unit exhibiting excellent low dielectric loss characteristics (low dissipation factor or low Df) and processability, a method for preparing the same, an epoxy composition, a cured product, and an article comprising the same.

According to a t aspect of the present disclosure, provided is a curing agent represented by formula (1).

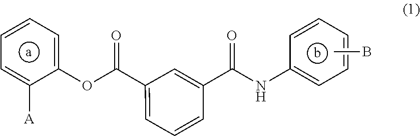

in which:
A is hydrogen or an allyl group, and
B is an OH group or a

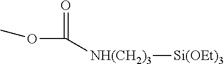

(where Et is an ethyl group) group.

According to a second aspect of the present disclosure, provided is a method for preparing a curing agent in which B is an OH group in formula (1), comprising: a first step of the formation of an ester group by mixing isophthaloyl chloride with at least one of phenol and 2-allyl phenol; and a second step of the formation of an amide functional group by mixing a reaction product of the first step with at least one of m-amino phenol, p-amino phenol and o-amino phenol,

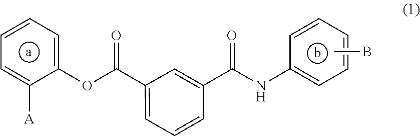

in which:
A is hydrogen or an allyl group, and
B is an OH group.

According to a third aspect of the present disclosure, provided is a method for preparing a curing agent in which B is a

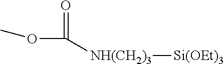

(where Et is an ethyl group) group in formula (1), the method comprising: a first step of the formation of an ester group by mixing isophthaloyl chloride with at least one of phenol or 2-allyl phenol; a second step of the formation of an amide functional group by mixing a reaction product of the first step with at least one of m-amino phenol, p-amino phenol or o-amino phenol; and a third step of the formation of an alkoxysilyl group by mixing a reaction product of the second step with 3-(triethoxysilyl) propyl isocyanate,

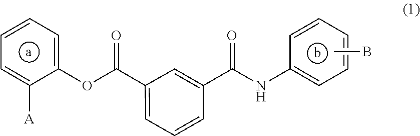

in which:
A is hydrogen or an allyl group, and B is a

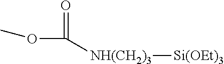

(where Et is an ethyl group) group.

According to a fourth aspect of the present disclosure, provided is the method according to the second aspect or the third aspect, wherein in the first step, at least one of phenol or 2-allyl phenol is mixed in an amount of 0.6 equivalents to 1.4 equivalents with respect to 1 equivalent of isophthaloyl chloride, and in the second step, the reaction product of the first step is mixed with at least one of m-amino phenol, p-amino phenol or o-amino phenol phenol in an amount of 0.6 equivalents to 1.4 equivalents with respect to one 1 equivalent of isophthaloyl chloride used in the first step.

According to a fifth aspect of the present disclosure, provided is the method according to the third aspect, wherein in the third step, 0.1 to 1.0 equivalents of 3-(triethoxysilyl) propyl isocyanate are mixed with respect to 1 equivalent of the hydroxyl group of the reaction product of the second step.

According to a sixth aspect of the present disclosure, provided is an epoxy composition comprising an (A) epoxy resin and a (B) curing agent, wherein as the (B) curing agent, (i) the curing agent represented by formula (1) of the first aspect is used alone, or (ii) the curing agent (a) represented by formula (1) of the first aspect and at least one curing agent (b) of an active ester-based curing agent and a phenol-based curing agent are used together.

According to a seventh aspect of the present disclosure, provided is the epoxy composition according to the sixth aspect, wherein when the curing agent (a) represented by formula (1) of the first aspect and at least one curing agent (b) of the active ester-based curing agent and the phenol-based curing agent are used together, the curing agent (a) and the curing agent (b) are used in a mole ratio of 0.5:9.5 to 9.5:0.5.

According to an eighth aspect of the present disclosure, provided is a cured product of the epoxy composition of the sixth aspect or the seventh aspect.

According to a ninth aspect of the present disclosure, provided is an article comprising the epoxy composition of the sixth aspect or the seventh aspect, or the cured product of the eighth aspect.

According to a tenth aspect of the present disclosure, provided is the article according to the ninth aspect, wherein the article is an insulating film, an epoxy film for semiconductor packaging, an adhesive film, a build-up film, a substrate film, or an epoxy molding material.

According to the present disclosure, a novel curing agent having an group amide group ester and an simultaneously attached to a benzene unit (hereinafter, referred to as a ‘novel curing agent’) is used as a curing agent for an epoxy resin. Improved low dielectric loss (Df) and processability (specifically, desmearability) are achieved by such an epoxy composition comprising the novel curing agent.

An epoxy composition of the present disclosure comprising the novel curing agent of the present disclosure is suitable for use as an insulating film, an epoxy film for semiconductor packaging, an adhesive film, a build-up film, a substrate film, and an epoxy molding material for semiconductor packaging, or the like due to such improved characteristics.

### DETAILED DESCRIPTION

According to the present disclosure, provided is a novel curing agent exhibiting excellent low dielectric loss properties and processability (specifically desmearability) in epoxy materials, a method for preparing the same, an epoxy composition comprising the novel curing agent, a cured product of the epoxy composition, and an article comprising the epoxy composition. Hereinafter, these will be described.

### A. A Novel Curing Agent Having an Ester Group and an Amide Group Simultaneously Attached to a Benzene Unit

According to the present disclosure, a novel curing agent having an ester group and an amide group simultaneously attached to a benzene unit represented by formula (1) is provided.

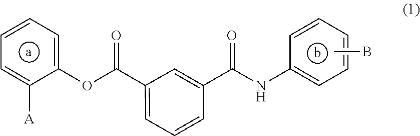

In formula (1),

A is hydrogen or an allyl group, and B is an OH group or a

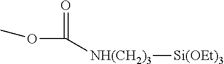

(where Et is an ethyl group) group. A substitution position of B in a benzene ring is not specified, and B may be substituted in any position of an ortho, meta, or para position, and may be preferably substituted in the meta or para position. Here, as a functional group connected to the ester group and the amide group, a benzene structure (\textcircled{a} benzene unit and \textcircled{b} benzene unit) is preferable. For example, when a structure is a naphthalene structure other than a benzene structure, solution dispersibility decreases, which is not preferable, and when the structure is an alkyl or an alicyclic structure, the properties such as dielectric loss decrease, which is not preferable.

In an application of an epoxy composition and/or an epoxy cured product comprising a novel curing agent according to the present disclosure, excellent low dielectric properties and processability (specifically desmearability) are accomplished by chemical structural properties of the novel curing agent according to the present disclosure. Due to the functional groups of the ester group and the amide group bonded to an central benzene unit in a meta position, and the structures of \textcircled{a} benzene and \textcircled{b} benzene, an molecular structure is optimized in terms of the balance of low dielectric properties and processability. In this aspect, it is not preferable that the ester group and the amide group are bonded to the central benzene unit in para and ortho positions. That is, a structure of the central benzene unit to which —CO— of the ester group and the amide group bonded is preferably an isophthaloyl unit structure, and it is not preferable that the structure of the central benzene unit is a terephthaloyl unit structure (a position of a carbonyl group with respect to the benzene ring being -para) and a phthaloyl unit structure (the position of the carbonyl group with respect to the benzene ring being -ortho).

The novel curing agent of formula (1) has a weight average molecular weight (Mw) range from 300 to 1,000 g/mol, more preferably 300 to 900 g/mol. It is preferable that Mw is in the above-described range in terms of material properties and processability. When Mw is less than 300 g/mol, this is not preferable in that the novel curing agent according to the present disclosure is not synthesized, and when Mw is more than 1,000 g/mol, this is not preferable in that it may be difficult to simultaneously secure dielectric properties and processability. Accordingly, a polymer having a repeating structure is not preferable. Here, Mw of the novel curing agent is a weight average molecular weight measured by gel permeation chromatography (GPC) method calculated based on polystyrene.

The novel curing agent of formula (1) has a curing agent equivalent (value obtained by dividing a solid mass of the curing agent by the number of functional groups that may react with the epoxy group) of 80 to 300 g/Eq, preferably 90 to 250 g/Eq. It is preferable that the curing agent equivalent is in the above-described range to ensure the material properties and processability, and when the curing agent equivalent is less than 80 g/Eq or more than 300 g/Eq, this is not preferable in that the dielectric properties and processability are not secured. During a curing reaction with an epoxy resin, the novel curing agent is dissociated into phenol, aminophenol, and isophthaloyl moiety to participate in the curing reaction.

The ester group and the amide group bonded to the benzene unit of the novel curing agent of formula (1) according to the present disclosure may suppress and/or prevent the formation of secondary alcohol during the epoxy curing reaction, thereby improving the dielectric loss properties. Additionally, due to the presence of the ester group and the amide group at the same time, the affinity of the curing agent for the solvent may be improved, and accordingly, solubility may be improved, thereby improving the dispersibility of the epoxy composition, as well as the enhanced desmearability and the solution dispersibility.

Furthermore, it exhibits improved low dielectric loss and processability (specifically, desmearability) by the balanced molecular architecture of the ester group and amide group bonded to the benzene unit.

When present, alkoxysilyl groups not only contribute to the improvement of dielectric loss (i.e., low dielectric loss properties) of an epoxy cured product by suppressing the formation of secondary alcohol, but also to improvement of the desmearability.

### B. A Method for Preparing the Novel Curing Agent

The novel curing agent according to the present disclosure is prepared by comprising (1) the formation of an ester group (first step); the formation of an amide group (second step); and optionally the formation of an alkoxysilyl group (third step).

As illustrated in the following reaction scheme 1, in the first step, an ester functional group is formed on the benzene unit by a reaction of isophthaloyl chloride with at least one of phenol and 2-allyl phenol.

[Reaction Scheme 1: Reaction Between Isophthaloyl Chloride and 2-allyl Phenol (the First Step)]

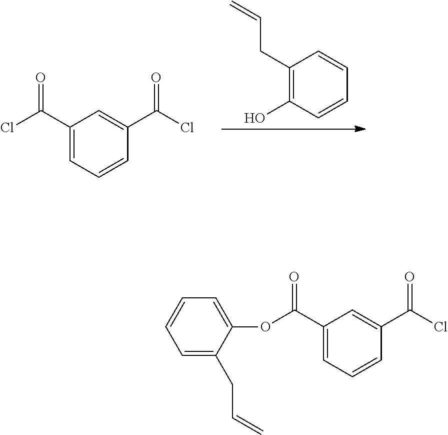

It is not limited thereto, but for example, a reaction of reactants, isophthaloyl chloride and at least one of phenol and 2-allyl phenol (hereinafter, referred to as “phenols”), may be carried out by contact such as mixing isophthaloyl chloride and phenols. The mixing is not limited thereto, but may be carried out, for example, by stirring of the reactants.

In the first step, isophthaloyl chloride and phenols are reacted with 0.6 equivalents to 1.4 equivalents, preferably 1 equivalent to 1.4 equivalents of the phenols with respect to 1 equivalent of isophthaloyl chloride (Here, 1 equivalent of isophthaloyl chloride corresponds to two equivalents based on acyl chloride, which is a reactive functional group). When the phenols are less than a lower limit, this is not preferable in that sufficient ester groups are not formed, and when the phenols are more than upper limit, this is not preferable in that the formation of amide groups in a following second step reaction is hindered.

In consideration of the solubility of the reactants and the reaction time, the first step may be carried out at a temperature ranging from 0° C. to 80° C., preferably 25° C. to 60° C. When the temperature is less than 0° C., the reaction rate may be significantly slowed, which is not preferable, and when the temperature is more than 80° C., this is not preferable in that a side reaction may occur.

The reaction time depends on the structure of the reactants, the degree of reaction, the amount of solvent, and the amount of base, but it is preferable that reaction time is 1 to 48 hours, preferably 1 to 24 hours, in terms of reaction efficiency. When the reaction time is less than 1 hour, this is not preferable in that it is difficult to allow the sufficient progress of the reaction, and a reaction time more than 48 hours is not preferable, in that additional reactions may occur.

Meanwhile, the reaction of isophthaloyl chloride with phenol and/or 2-allyl phenol may be performed in the presence of optional base and/or optional solvent.

If a separate base is used, it serves either to neutralize an acid generated during a reaction, or to accelerate the reaction rate. Examples of usable bases are not limited thereto, but may include NaOH, KOH, K₂CO₃, Na₂CO₃, KHCO₃, NaHCO₃, triethylamine, and diisopropyl ethylamine. These bases may be used alone or in combination of two or more thereof. When using the base, with respect to 1 equivalent of isophthaloyl chloride (2 equivalents based on acylchloride), 2 equivalents to 10 equivalents, preferably 2 equivalents to 4 equivalents, are suitable in terms of reaction efficiency. When a base content is less than 2 equivalents, neutralization of an acid generated during the reaction may not be sufficient, and even if the base content exceeds 10 equivalents, the effect of the base addition is not further improved, and a larger amount of use of the base is unnecessary.

Additionally, the solvent may be optionally used as needed. For example, in the reaction of the first step, when the viscosity of the reactants at a reaction temperature is suitable for the reaction to proceed without a separate solvent, the solvent may not be used. That is, when the viscosity of the reactants is lowered so that the mixing and stirring of the reactants may proceed smoothly without a solvent, a separate solvent is not required, which may be easily determined by those skilled in the art. In the case of using the solvent, any solvent may be used as long as the solvent may dissolve the reactants properly, and may be easily removed after the reaction without any adverse effect on the reaction. Examples of the solvents may include toluene, xylene, acetonitrile, tetrahydrofuran (THF), methyl ethyl ketone (MEK), dimethyl formamide (DMF), dimethyl sulfoxide (DMSO), methylene chloride (MC), chloroform (CHCl₃), and water (H₂O), or the like, but the present disclosure is not limited thereto. These solvents may be used alone or in combination of two or more thereof. The amount of the solvent used is not particularly limited, and a suitable amount thereof may be used in a range in which the reactants are sufficiently dissolved and do not adversely affect the reaction, and those skilled in the art may consider this and select the content of solvent appropriately.

As illustrated in the following reaction scheme 2, in the second step, an amide functional group is formed on the benzene unit by reaction between the reaction product of the first step and amino phenol.

### [Reaction Scheme 2: Reaction Between the Reaction Product in the First Step and Amino Phenol (the Second Step)]

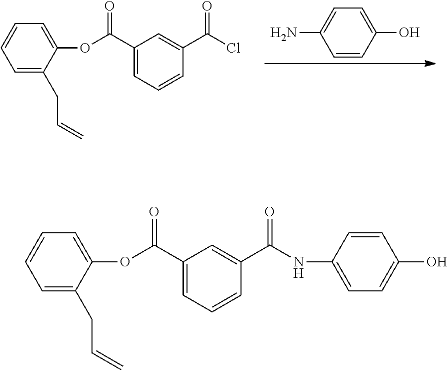 [SMILES: C=CCc1ccccc1OC(=O)c1cccc(C(=O)Cl)c1 ; Nc1ccc(O)cc1 ; C=CCc1ccccc1OC(=O)c1cccc(C(=O)Nc2ccc(O)cc2)c1]

In the second step, the reaction of the reaction product of the first step with aminophenol (m-aminophenol, p-aminophenol and/or o-aminophenol) is not limited thereto, but may be performed by contact, such as, for example, mixing the reaction product of the first step with aminophenol. The mixing is not limited thereto, but may be, for example, performed by stirring the reactants.

In the reaction of the second step, 0.6 to 1.4 equivalents of aminophenol, preferably 0.6 to 1 equivalent, may be used for 1 equivalent of isophthaloyl chloride (2 equivalents based on acyl chloride) used as a reactant in the first step. When the amount of aminophenol is less than 0.6 equivalents, this is not preferable in that some of the isophthaloyl chloride may not react, which may cause a side reaction to occur, and when the amount of aminophenol exceeds 1.4 equivalents, this is not preferable in that the purification time may increase and byproducts may be included.

In consideration of the solubility of the reactants and the reaction time, the second step may be performed at a temperature ranging from 0° C. to 80° C., preferably 25° C. to 60° C. When the temperature is less than 0° C., this is not preferable in that the reaction rate may be significantly slowed, and the temperature exceeding 80° C., is not preferable, in that the side reaction may occur.

The reaction time varies depending on the structure of the reactants, the degree of reaction, the amount of solvent and base, but 1 hour to 48 hours of reaction time is desirable in terms of reaction efficiency, preferably 1 hour to 24 hours. When the reaction time is less than 1 hour, this is not preferable in that it is difficult for the reaction to proceed sufficiently, and a reaction time more than 48 hours, is not preferable, in that the side reaction may occur.

Meanwhile, the reaction of the first step reaction product with aminophenol may be performed in the presence of optional base and/or optional solvent.

When a separate base is used, the base may be used to neutralize the acid generated during the reaction or to speed up the reaction rate. Examples of usable bases may include, but are not limited to, NaOH, KOH, K₂CO₃, Na₂CO₃, KHCO₃, NaHCO₃, triethylamine, and diisopropylethylamine. These bases may be used alone or in combination of two or more thereof. If the base is used, with respect to 1 equivalent of isophthaloyl chloride (2 equivalents based on acyl chloride), 2 equivalents to 10 equivalents, preferably 2 equivalents to 4 equivalents of the base, are suitable in terms of reaction efficiency. When a base content is less than 2 equivalents, an intended effect of base addition may be insufficient, and even if the base content is used in excess of 10 equivalents, the effect of the base addition is not further improved, and a large amount of use of the base is unnecessary.

Additionally, the solvent may be used optionally as needed. For example, in the reaction of the second step, if the viscosity of the reactants at the reaction temperature is suitable for the reaction to proceed without a separate solvent, the solvent may not be used. That is, when the viscosity of the reactant is lowered so that the mixing and stirring of the reactants may proceed smoothly without a solvent, a separate solvent is not required, which may be easily determined by those skilled in the art. In the case of using the solvent, any solvent may be used as long as the solvent may dissolve the reactants properly, and may be easily removed after the reaction without any adverse effects on the reaction. Examples of the solvents may include toluene, xylene, acetonitrile, tetra hydro furan (THF), methyl ethyl ketone (MEK), dimethyl formamide (DMF), dimethyl sulfoxide (DMSO), methylene chloride (MC), chloroform (CHCl₃), and water (H₂O), but the present disclosure is not limited thereto. These solvents may be used alone or in combination of two or more thereof. The amount of the solvent used is not particularly limited, and a suitable amount thereof may be used in a range in which the reactants are sufficiently dissolved and do not adversely affect the reaction, and those skilled in the art may consider this and select the content of solvent appropriately.

By the completion of the reaction of the second step, a novel curing agent in which A is hydrogen or an allyl group and B is an OH group in formula (1) according to the present disclosure is obtained.

As is generally known in the chemical field, not all reactants in a chemical reaction are 100% completely reacted, and the reaction product may comprises a final target intended herein (i.e., the novel curing agent represented by formula (1)), and depending on various factors such as the reactivity of the reactants, starting materials, products which is obtained by completely proceeding the reaction, products which is obtained by partially proceeding the reaction, or the like, may be presented together, and the reaction product in which these various materials are presented together (hereinafter, referred to as the ‘final product’ for convenience) may be used as a novel curing agent represented by formula (1) in an epoxy composition as it is.

Meanwhile, the physical properties and/or processability (specifically, desmearability) expressed by the novel curing agent in the final product obtained after the completion of the second step may be controlled by adjusting the amount of phenols used in the first step and aminophenols used in the second step (i.e., the equivalent ratio). For example, to achieve the balance between solution dispersibility and physical properties (thermal properties, glass transition temperature, or the like) by the novel curing agent, the amount of phenols used in the first step and aminophenols used in the second step may be controlled to be in the range of 7:3 to 3:7 equivalent ratio (molar ratio) of phenols:aminophenols, more preferably 6:4 to 4:6 equivalent ratio, and most preferably 5:5 equivalent ratio. For example, in order to increase the dielectric properties (i.e., low dielectric loss properties) in the epoxy composition, the concentration of phenols may be increased, and in order to further increase the physical properties (resistance to heat expansion properties, etc.), the concentration of aminophenols may be increased. When the content of aminophenol is more than the above-described range, the dielectric properties may deteriorate because the content of phenols is relatively low, and when the content of aminophenol is less than the above-described range, the thermal properties may deteriorate.

As illustrated in the following reaction scheme 3, in the third step, an alkoxysilyl group is formed by the reaction of the reaction product of the second step with an isocyanate silane coupling agent, 3-(triethoxysilyl) propyl isocyanate (i.e., OCN—(CH₂)₃—Si(OEt)₃). Meanwhile, the third step is an optional step that may be performed when an alkoxysilyl group is to be formed.

[Reaction Scheme 3: Reaction of the Reaction Product of the Second Step with 3-(triethoxysilyl) Propyl Isocyanate (the Third Step)]

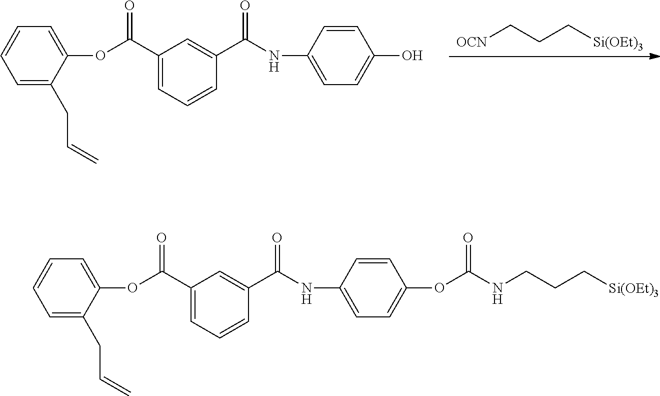

The present disclosure is not limited thereto, but for example, in the third step, the reaction of the reaction product of the second step with 3-(triethoxysilyl) propyl isocyanate (i.e., OCN—(CH₂)₃—Si(OEt)₃) may be performed by contact, such as mixing the reaction product of the second step with 3-(triethoxysilyl) propyl isocyanate (i.e., OCN—(CH₂)₃—Si(OEt)₃). The mixing is not limited thereto, but may be performed, for example, by stirring the reactants.

In the third step, the OH group of the reaction product of the second step and 3-(triethoxysilyl) propyl isocyanate react, in terms of the silylation ratio and reaction efficiency, 0.1 to 1.0 equivalents of 3-(triethoxysilyl) propyl isocyanate is added for 1 equivalent of the hydroxyl group of the reaction product of the second step. When 3-(triethoxysilyl) propyl isocyanate is less than 0.1 equivalent, this may be insufficient for the silylation reaction, and when 3-(triethoxysilyl) propyl isocyanate is added by exceeding 1.0 equivalent, this is unnecessary because the reaction has already progressed sufficiently.

The third step reaction may be performed at a reaction temperature ranging from 60° C. to 150° C., preferably 80° C. to 120° C. In consideration of the solubility of the reactants and the reaction time in the reaction process, the reaction temperature may be 60° C. to 150° C., and when the reaction temperature is less than 60° C., unreacted reactants may remain, and when the reaction temperature exceeds 150° C., this is not preferable in that the side reaction may occur due to the high reaction rate.

The reaction time of the third step is 1 hour to 72 hours, preferably 12 hours to 24 hours, and may vary depending on the structure of the reactants, the degree of reaction, the amount of the solvent, and the base. When the reaction time is less than 1 hour, the reaction may not proceed sufficiently, and even if the reaction time exceeds 72 hours, this is not preferable in that the reaction does not proceed further.

The reaction of the third step may be performed in the presence of optional base and/or optional solvent.

That is, the reaction of the third step may be performed in the presence of a base as needed. When a separate base is used, the reaction rate may be increased. Examples of usable bases are not limited thereto, but may include triethylamine, diisopropylethylamine, and pyridine, or the like. These bases may be used alone or in combination of two or more thereof. When using the base, the base in an amount of 0.1 to 5 equivalents with respect to 1 equivalent of the hydroxyl group of the reaction product of the second step may be properly used in terms of reaction efficiency. When the base content is less than 0.1 equivalent, an intended effect of the base addition may be insufficient, and even if the base content is used by exceeding 5 equivalents, the effect of the base addition is not further improved, and a larger amount of use of the base is unnecessary.

Additionally, the solvent may be used optionally as needed. For example, in the reaction of the third step, when the viscosity of the reactants at the reaction temperature is suitable for the reaction to proceed without a separate solvent, the solvent may not be used. In other words, when the viscosity of the reactant is lowered so that the mixing and stirring of the reactants may proceed smoothly without a solvent, a separate solvent is not required, which may be easily determined by those skilled in the art. In the case of using the solvent, any solvent may be used as long as the solvent may dissolve the reactants properly, and may be easily removed after the reaction without any adverse effects on the reaction. For example, toluene, xylene, acetonitrile, tetra hydro furan (THF), methyl ethyl ketone (MEK), dimethyl formamide (DMF), dimethyl sulfoxide (DMSO), methylene chloride (MC), chloroform (CHCl₃), water (H₂O), or the like, may be used as the solvents, but the present disclosure is not limited thereto. These solvents may be used alone or in combination of two or more thereof. The amount of the solvent is not particularly limited, and a suitable amount thereof may be used in a range in which the reactants are sufficiently dissolved and do not adversely affect the reaction, and those skilled in the art may consider this and select the content of solvent appropriately.

As is generally known in the chemical field, not all reactants in a chemical reaction are 100% completely reacted, and thus, the reaction product comprises the novel curing agent intended in the present disclosures, and depending on various factors such as reactivity of reactants, starting materials, products which is obtained by completely proceeding the reaction, products which is obtained by partially proceeding the reaction, or the like, may be presented together, and the reaction products in which these various materials are presented together may be used as a curing agent in the epoxy composition as it is.

### C. Epoxy Composition

In another embodiment of the present disclosure, an epoxy composition is provided that not only exhibits improved low-dielectric loss property in an epoxy cured product, but also exhibits excellent processability (specifically, desmearability), and the epoxy composition of the present disclosure comprises an (A) epoxy resin, and a (B) curing agent.

The epoxy composition of the present disclosure may additionally include, if necessary, one or more of (C) an inorganic filler, (D) a thermoplastic resin, and (E) a curing catalyst. Hereinafter, the components of the epoxy composition of the present disclosure will be described respectively.

### (A) Epoxy Resin

An epoxy composition of the present disclosure comprises an epoxy resin (A) as a main resin. The epoxy resin (A) may be any epoxy resin conventionally known in the technical field, and the type and/or properties thereof are not limited. Details on the epoxy resin are generally known in the technical field and are not described in detail herein.

General epoxy resins are not limited thereto, but examples of the epoxy resins may include a glycidyl based epoxy resin selected from the group consisting of a glycidyl amine-based epoxy resin, a glycidyl ester-based epoxy resin, and a glycidyl ether-based epoxy resin having bisphenol, biphenyl, naphthalene, benzene, thiodiphenol, fluorene, anthracene, isocyanurate, triphenylmethane, 1,1,2,2-tetraphenylethane, tetraphenylmethane, 4,4′-diaminodiphenylmethane, aminophenol, an alicyclic, aliphatic or novolak unit, and an alicyclic based epoxy resin, and at least one selected from these epoxy resins may be used. The general epoxy resins include both liquid and solid epoxy resins. The general epoxy resins may also include epoxy resins having an alkoxysilyl group in addition to the epoxy group.

In this technical field, in consideration of an epoxy composition, a cured product thereof and an application field thereof, physical properties such as processability, drying properties and viscosity in a subsequent process of processing the epoxy composition, various epoxy resins may be generally mixed and used together. Accordingly, those skilled in the art may appropriately mix and use various general epoxy resins as needed, in consideration of the physical properties required according to the application field of the epoxy composition, which is not described in detail in this specification.

The epoxy equivalent of the above epoxy resin is also not particularly limited, but may be, for example, 70 g/Eq to 1000 g/Eq, preferably 80 g/Eq to 1000 g/Eq, more preferably 80 g/Eq to 800 g/Eq, and even more preferably 100 g/Eq to 500 g/Eq. By utilizing the epoxy resin in the epoxy equivalent range, the crosslinking density of the cured product may be sufficient to secure the cured product properties.

The weight average molecular weight (Mw) of the epoxy resin is also not particularly limited, but may be, for example, 300 to 8000 g/mol, preferably 300 to 5000 g/mol, and more preferably 300 to 4000 g/mol in terms of processability and compatibility with inorganic fillers. Here, the weight average molecular weight of the epoxy resin is a weight average molecular weight measured by gel permeation chromatography (GPC) method using polystyrene.

### (B) Curing Agent

An epoxy composition of the present disclosure comprises a curing agent (B), and as the curing agent, \textcircled{1} a novel curing agent represented by formula (1) of the present disclosure described above may be used alone, or (2) a novel compound represented by formula (1) of the present disclosure described above and a conventional curing agent may be used together.

Since the novel curing agent represented by formula (1) of the present disclosure is used in the epoxy composition of the present disclosure, all of the contents of the novel curing agent having the ester group and the amide group on the benzene unit described in item A above are equally applied.

The conventional curing agents that may be used together with the novel curing agent are an active ester-based curing agent and/or a phenol-based curing agent.

In terms of the low dielectric properties of the cured product of the epoxy composition of the present disclosure, the novel curing agent and the conventional curing agent may be used together. A cured product of the epoxy composition in which the novel curing agent is used alone exhibits a dielectric loss of less than 0.007, preferably less than 0.006. A cured product of the epoxy composition in which the novel curing agent and the conventional curing agent are used together exhibits a dielectric loss of less than 0.005, preferably less than 0.003.

Any active ester-based curing agent known to be conventionally used for epoxy resins may be used as the active ester-based curing agent. For example, as the active ester-based curing agent, a compound having two active ester groups per a molecule is preferable, but is not limited thereto. An active ester compound obtained by a reaction between a carboxylic acid compound and a hydroxy compound is preferable. Here, isophthalic acid, terephthalic acid, or the like, are preferable as the carboxylic acid compound used to produce the active ester-based curing agent, and phenol, naphthol, or the like, are preferable as the hydroxy compound. The active ester-based curing agents may be used alone or in any combination of two or more types thereof.

Any phenol novolac-based curing agent known to be conventionally used for epoxy resins may be used as the phenol-based curing agent. For example, phenol-based curing agents include, but are not limited to, a phenol novolac resin, a trifunctional phenol novolac resin, a cresol novolac resin, a bisphenol A novolac resin, a xylene novolac resin, a triphenyl novolac resin, a biphenyl-based novolac resin, a dicyclopentadiene novolac resin, a naphthalene-based novolac resins, a phenol p-xylene resins, a phenol 4,4′-dimethylbiphenylene resin, xylok (modified p-xylene), and a triazine-based compound, or the like.

Additionally, the above-mentioned phenolic-based curing agents may additionally have an alkoxysilyl group in addition to the phenol group. Accordingly, the above-mentioned phenolic-based curing agent also includes all phenolic-based curing agents having a phenol group and additionally an alkoxysilyl group. The above-mentioned phenolic-based curing agents may be used alone or in any combination of two or more types thereof.

The amount of the curing agent in the epoxy composition is determined by an equivalent ratio with respect to the epoxy resin. The present disclosure is not limited thereto, but for example, the curing agent is used in the range of 0.7 to 1.4 equivalents, preferably 0.7 to 1.3 equivalents, with respect to 1 equivalent of the epoxy resin. When the curing agent is less than 0.7 equivalents, sufficient dielectric loss characteristics may not be secured, and when the curing agent exceeds 1.4 equivalents, the curing efficiency of the epoxy cured product may decrease.

The amount of the curing agent is an amount of the novel curing agent when the novel curing agent is used alone, and the amount of the curing agent is the sum of the amount of the novel curing agent and the amount of the conventional curing agent when the novel curing agent and the conventional curing agent are used together as curing agents.

Here, the equivalent of the epoxy resin is a value obtained by dividing a solid mass of the epoxy resin by the number of epoxide functional groups, and the equivalent of the curing agent is a value obtained by dividing a solid mass of the curing agent by the ‘number of functional groups that may react with the epoxy group.’ The equivalent of the curing agent is the sum of all curing agents used in the formulation. Accordingly, when the novel curing agent is used alone, an equivalent thereof is an equivalent of the novel curing agent, and when the novel curing agent and the conventional curing agent are used together as curing agents, an equivalent thereof is an equivalent of the sum of the amount of the novel curing agent and the amount of the conventional curing agent. Here, the ‘solid mass’ refers to a weight of a pure epoxy resin and a curing agent excluding any incidental liquids and solvents that may exist in the epoxy resin and the curing agent. A calculation of the equivalent of the epoxy resin and the curing agent is generally well known in the technical field.

Meanwhile, when the novel curing agent (a) and the conventional curing agent (b) (i.e., the active ester-based curing agent and/or the phenol-based curing agent) are used together, the novel curing agent (a) and the conventional curing agent (b) may be used together in a molar ratio of the novel curing agent (a) to conventional curing agent (b) of 0.5:9.5 to 9.5:0.5, preferably 9:1 to 1:9. That is, the novel curing agent (a) and the conventional curing agent (b) may be mixed at the above-described molar ratio, and the mixed curing agent may be used in the range of 0.7 to 1.4 equivalents, preferably 0.7 to 1.3 equivalents, with respect to 1 equivalent of the above-described epoxy resin.

In the molar ratio of the novel curing agent (a) and the conventional curing agent (b), when the novel curing agent is used below a lower limit, intended physical properties are not sufficiently secured using the novel curing agent, which is not preferable, and even if the novel curing agent exceeds the upper limit, an improvement effect by mixing the novel curing agent is no longer increased, which is not preferable.

When an active ester-based curing agent and a phenol-based curing agent are used together as the conventional curing agent, a mixing ratio thereof is not particularly limited, and in consideration of the physical properties of the intended cured product, the phenol-based curing agent and the active ester-based curing agent may be mixed and used at any suitable ratio, which is generally known in this technical field and is not described in detail here.

### (C) Inorganic Filler

An epoxy composition of the present disclosure may additionally include an inorganic filler as needed. The inorganic filler is a component generally used in this technical field to reinforce the properties of the epoxy composition.

For example, any inorganic filler known in this technical field to be used to reinforce the properties of conventional epoxy resins may be used as the inorganic filler. For example, the inorganic fillers may include silica (including, for example, fused silica and crystalline silica), zirconia, titania, alumina, metal oxides such as magnesium oxide, aluminum nitride, silicon nitride, and aluminum nitride, and silsesquioxane but not limited thereto. The inorganic fillers may be used alone or as a mixture of two or more thereof. An average particle diameter of the inorganic filler is not limited, but for example, the inorganic filler may use spherical powder particles having a particle size (cut size) of 0.01 μm to 100 μm, preferably 0.02 μm to 70 μm, more preferably 0.02 μm to 50 μm in terms of dispersibility, processability, and reliability. When the average particle diameter of the inorganic filler is less than 0.01 μm, this is not only expensive but may also have problems with dispersion, and if when exceeds 100 μm, this may cause problems with fine pattern formation and filling.

The inorganic filler is generally known in this technical field and is not be described in detail here.

The content of the inorganic filler in the epoxy composition of the present disclosure may be, for example, 93 wt % or less based on the total weight of a solids content of an epoxy composition (for example, 93 parts by weight or less with respect to 100 parts by weight of the solids content of the epoxy composition), from the viewpoint of using an amount necessary to secure the physical properties required in the epoxy composition. Since the inorganic filler may be mixed as needed, a lower mixing amount is not particularly limited, but when the inorganic filler is added, in order to show the effect by the addition, the inorganic filler may be mixed at least 10 wt % (for example, 10 parts by weight with respect to 100 parts by weight of the solids content of the epoxy composition). In consideration of the securing of physical properties and dispersibility by the epoxy composition, or the like, the inorganic filler may be used in an amount of, for example, 10 wt % to 93 wt %, preferably 20 wt % to 90 wt %, in the above-described content. When the amount is less than 10 wt %, this is not preferable in that it is difficult to secure the physical properties of the intended epoxy cured product by mixing the filler, and when the amount exceeds 93 wt %, it is not preferable in that it is difficult to disperse the filler.

### (D) Thermoplastic Resin

The epoxy composition of the present disclosure may additionally comprise a thermoplastic resin as needed. For example, the thermoplastic resin may be additionally used when it is necessary to impart film properties and/or flexibility.

As the thermoplastic resin, any thermoplastic resin generally known to be used in this technical field, such as an acrylic resin, a phenoxy resin, a polyvinyl acetal resin, a polyimide resin, a polyamideimide resin, a polyether sulfone resin, a polysulfone resin, or the like, may be used alone or in combination of two or more thereof, and the structure, the molecular weight of the thermoplastic resins, or the like are not limited. The details of the thermoplastic resin are generally known in this technical field and are not described in detail herein.

When the thermoplastic resin is used, the thermoplastic resin may be used in an amount generally used in this technical field and is not particularly limited. However, for example, the thermoplastic resin is a component that may be mixed as needed, and may be mixed at 7 wt % or less (i.e., 7 parts by weight or less per 100 parts by weight of the total solids content of the epoxy composition) based on the total solids content of the epoxy composition. However, when the thermoplastic resin is mixed, in consideration of the processability (film formability) and/or flexibility of the epoxy composition, the thermoplastic resin may be mixed and used at 0.5 wt % to 7 wt % (i.e., 0.5 to 7 parts by weight per 100 parts by weight of the total solids content of the epoxy composition) based on the total solids content of the epoxy composition to exhibit the intended effect. When the thermoplastic resin is added at less than 0.5 wt %, this is not preferable in that the intended properties of the thermoplastic resin, such as film processability or flexibility, are not sufficient, and when the thermoplastic resin is added by exceeding 7 wt %, it is not preferable in that the properties of the epoxy material may deteriorate.

### (E) Curing Catalyst

The epoxy composition of the present disclosure may also include a curing catalyst. By rapidly curing the epoxy composition using the curing catalyst, the crosslinking structure in the cured product becomes uniform and the crosslinking density increases.

The curing catalyst is not particularly limited, and any curing catalyst generally known in this technical field to be used as a curing catalyst for curing epoxy resins may be used. The curing catalyst may be used alone or in combination of two or more thereof.

For example, imidazole-based compounds, amine-based compounds, organophosphine-based compounds, organophosphonium salt-based compounds, or the like, may be used as the curing catalyst.

Examples of the imidazole-based compounds may include 2-undecylimidazole, 2-heptadecylimidazole, 2-methylimidazole, 2-ethyl-4-methylimidazole, 2-phenylimidazole, 2-phenyl-4-methylimidazole, 1-benzyl-2-methylimidazole, 1-benzyl-2-phenylimidazole, 1,2-dimethylimidazole, 1-cyanoethyl-2-methylimidazole, 1-cyanoethyl-2-ethyl-4-methylimidazole, 1-cyanoethyl-2-undecylimidazole, 1-cyanoethyl-2-phenylimidazole, 1-cyanoethyl-2-undecylimidazolium trimellitate, 1-cyanoethyl-2-phenylimidazolium trimellitate, 2,4-diamino-6-[2′-methylimidazolyl-(1′)]-ethyl-s-triazine, 2,4-diamino-6-[2′-undecylimidazolyl-(1′)]-ethyl-s-triazine, 2,4-diamino-6-[2′-ethyl-4′-methylimidazolyl-(1′)]-ethyl-s-triazine, 2,4-diamino-6-[2′-methylimidazolyl-(1′)]-ethyl-s-triazineisocyanuric acid 2-adduct, phenylimidazoleisocyanuric acid adduct, 2-methylimidazoleisocyanuric acid adduct, 2-phenyl-4,5-dihydroxymethylimidazole and 2-phenyl-4-methyl-5-dihydroxymethylimidazole, 1,3,5-triazine-2,4-diamine6-[2-(2-methyl-1H-imidazol-1-yl)ethyl] and 1,3,5-triazine-2,4,6(1H,3H,5H)-trione (1:1), or the like.

Examples of the amine-based compounds may include diethylamine, triethylamine, diethylenetetramine, triethylenetetramine, 4-dimethylaminopyridine (DMAP), benzyl dimethyl amine (BDMA), trisdimethylaminomethylphenol (DMP-30), triethylenediamine, and diazabicycloundecene (DBU), or the like.

As the organic phosphine-based compounds and organic phosphonium salt-based compounds, TPP, TPP-K, TPP-S, TPTP-S, TBP-DA, TPP-SCN, TPTP-SCN (product name of Hokkyo Chemical Industry Co., Ltd.) may be used alone or in combination of two or more.

The curing catalyst may be used in an amount generally used in this technical field. The curing catalyst may be used in an amount of, for example, 0.1 to 10 parts by weight, for example, 0.2 to 10 parts by weight per 100 parts by weight of the epoxy resin, but not limited thereto. When the amount of the curing catalyst used is less than 0.1 parts by weight, the effect due to the use of the curing catalyst is insignificant, and when the amount of the curing catalyst exceeds 10 parts by weight, there is no additional improvement effect, which is not preferable. The curing catalyst is preferably used in the amount in terms of the effect of promoting the curing reaction and controlling the curing reaction speed. By using the curing catalyst in the range of the above, the curing operation may be advanced quickly and an improvement in throughput may be expected.

### (F) Other Additives

In the epoxy composition according to any aspect of the present disclosure may, within a range that does not damage the properties of the epoxy composition, other additives such flame as retardants, plasticizers, antibacterial agents, leveling agents, antifoaming agents, colorants, stabilizers, coupling agents, viscosity regulators, diluents, and molding agents that are commonly incorporated into an epoxy composition in this technical field for the purpose of controlling the properties of the epoxy composition may also be used as needed. Additionally, the epoxy composition may be dispersed using a solvent as needed so that the components thereof may be easily dispersed before curing. The types, formulations, and contents of these other additives and/or solvents are generally known to those skilled in the art, and are not described in detail herein.

In the present disclosure, the ‘solids content of the epoxy composition’ refers to the content of component(s) of the epoxy composition cured to form a final product (e.g., a cured product), excluding any liquid component such as solvent and liquid component(s) that are removed during drying and/or curing, in a case in which a liquid component(s) is accidentally present in the epoxy composition and/or the solvent is used (even if the component is a liquid component, the component comprised in the final product is comprised in the solids content). For example, in the case of an epoxy composition comprising an (A) epoxy resin, and a (B) curing agent, and optional (C) an inorganic filler, (D) a thermoplastic resin, (E) a curing catalyst, and/or (F) other additives, the ‘solids content of the epoxy composition’ refers to the total weight of the components of the epoxy composition cured to form the final product (e.g., a cured product) (even if the component is a liquid component, the component comprised in the final product is comprised in the solids content), excluding any liquid component such as a solvent removed during drying and/or curing, among the epoxy composition comprising the above.

Additionally, the ‘composition of the present disclosure’ in the above and below is understood to refer to an epoxy composition that essentially comprises an (A) epoxy resin and a (B) curing agent, and optionally comprises (C) an inorganic filler, (D) a thermoplastic resin, (E) a curing catalyst, and/or (F) other additives, if necessary.

For example, in the epoxy composition of the present disclosure, (B) the curing agent may be comprised in an amount ranging from 0.7 to 1.4 equivalents based on 1 equivalent of the epoxy resin, (C) the inorganic filler may be comprised in an amount of 93 wt % or less, preferably 10 wt % to 93 wt %, based on the total weight of the epoxy composition, and (D) the thermoplastic resin may be comprised in an amount of 7 wt %, preferably 0.5 wt % to 7 wt %, based on the total weight of the epoxy composition. The epoxy composition may also comprise, if necessary, (E) a curing catalyst and (F) other additives in amounts commonly used in this technical field. In the epoxy composition according to the present disclosure, the remainder of the content (for example, the remainder based on 100 wt %) may be an epoxy resin.

Additionally, the epoxy composition of the present disclosure comprises these components in the above-described mixing ratio range so that the total sum is 100 wt % based on the solids content. When the composition of the components (for example, components (C) and (D)) expressed in wt % based on the total weight of the solids content of the epoxy composition does not reach 100 wt %, the remainder is composed of other components (specifically, (A) the epoxy resin, (B) the curing agent, (E) the curing catalyst, and/or (F) other additives) to reach 100 wt %.

### D. Cured Product and Use of Epoxy Composition

According to another embodiment of the present disclosure, a cured product of the epoxy composition according to the present disclosure is provided. The cured product may be obtained by curing the epoxy composition, for example, by thermal curing. Those skilled in the art may appropriately select any curing method and curing conditions generally known in the pertinent art to cure the epoxy composition, and the curing method and/or curing conditions are not particularly limited.

Additionally, the curing method and curing conditions, or the like of the epoxy composition are generally known in this technical field and are not described in detail herein. The cured product is a partially cured and fully cured epoxy cured product, and is used to refer to both an epoxy cured product without an inorganic filler and an epoxy composite comprising a filler. As described above, the cured product formed by the epoxy composition of the present disclosure has improved processability (specifically, desmearability) as well as improved low dielectric loss characteristics.

For example, the cured product of the epoxy composition comprising the novel curing agent of the present disclosure exhibits a dielectric loss of less than 0.010, preferably less than 0.009, and more preferably less than 0.008. Additionally, the cured product of the epoxy composition comprising the novel curing agent of the present disclosure has a glass transition temperature of 120° C. or higher, preferably 130° C. or higher.

Furthermore, according to another embodiment of the present disclosure, an article comprising the epoxy composition and/or the cured product thereof according to any aspect of the present disclosure is provided.

As described above, the epoxy composition comprising the novel curing agent of the present disclosure achieves not only low dielectric loss but also excellent processability, i.e., desmearability in the epoxy cured product, and is suitable for an insulating film, an epoxy film for semiconductor packaging, an adhesive film, a build-up film, a substrate film, an epoxy molding material, and specifically, for next-generation semiconductor packaging, for example, packaging for component that processes large amounts of data at ultra-high speed (advanced semiconductor packaging).

Examples of the article may include an epoxy insulating film, specifically an insulating film for an IC substrate, an epoxy film for semiconductor packaging, an adhesive film, a build-up film, a substrate film, or an epoxy molding material such as an epoxy molding compound for semiconductor packaging, an underfill such as a next-generation semiconductor packaging component, for example, a packaging component that processes a large amount of data at an ultra-high speed, or the like.

A method for preparing an epoxy insulating film, for example, an insulating film for an IC substrate, an epoxy film for semiconductor packaging, an adhesive film, a build-up film, a substrate film or an epoxy molding material such as an epoxy molding compound for semiconductor packaging, an underfill such as a next-generation semiconductor packaging component, for example, a packaging component that processes a large amount of data at an ultra-high speed using the epoxy composition of the present disclosure, is not particularly limited, and the article above may be manufactured by any method known in the technical filed, and the method therefor is not described in detail herein.

For example, the film-shaped article may be prepared in a film shape by applying the epoxy composition of the present disclosure to a release film, and drying and curing the epoxy composition through a film forming process or in a film shape through extrusion of an epoxy composition. Various methods known in the pertinent field may be used as the film forming process, and examples thereof may include various printing methods such as offset printing and screen printing, a blade coating method, a dip coating method, a spin coating method, a bar coating method, a slit coating method, an inkjet method, and a die coating method, but the present disclosure is not limited thereto.

### EXAMPLES

Hereinafter, the present disclosure will be described in more detail through examples. However, the examples are intended to illustrate the present disclosure and are not intended to limit the present disclosure.

### A. Synthesis Example

### (1) Synthesis Example 1

At room temperature (15° C. to 25° C., the same applies hereinafter), 15 g of isophthaloyl chloride and 150 g of chloroform were added to a two-necked flask and were stirred at room temperature under a nitrogen atmosphere. Then, 9.91 g of allylphenol and 9.55 g of N,N-diisopropylethylamine (DIPEA) were sequentially added for 30 minutes, and were stirred and mixed for an additional 2 hours to synthesize the reaction product of the first step having an ester functional group.

To the reaction product obtained in the first step, 8.06 g of 3-aminophenol and 19.10 g of N,N-diisopropylethylamine (DIPEA) were sequentially added for 30 minutes at room temperature. Then, the reaction of the step was performed by stirring for 10 hours at room temperature. After the reaction was completed, a base and a solvent were removed using a rotary evaporator, and then, the reaction product was dried using a vacuum pump, thereby synthesizing a novel curing agent having an ester group and an amide group bonded to a benzene unit (curing agent equivalent: 94 g/Eq, [ester group]:[amide group]=5.0:5.0 by mol).

### (2) Synthesis Example 2

A novel curing agent having an ester group and an amide group (curing agent equivalent: 103 g/Eq, [ester group]:[amide group]=7:3 by mol) was synthesized in the same manner as in Synthesis Example 1, except that 9.73 g of phenol and 13.37 g of N,N-diisopropylethylamine (DIPEA) were used in the reaction of the first step, and 4.84 g of 3-aminophenol was used in the reaction of the second-step.

### (3) Synthesis Example 3

At room temperature, 15 g of isophthaloyl chloride and 150 g of chloroform were added to a two-necked flask and were stirred at room temperature under nitrogen. Then, 5.95 g of allylphenol and 5.73 g of N,N-diisopropylethylamine (DIPEA) were sequentially added at room temperature for 30 minutes and then mixed for an additional 2 hours, thereby synthesizing a reaction product of the first step having an ester functional group.

To the reaction product obtained in the first step, 11.29 g of 4-aminophenol and 19.10 g of N,N-diisopropylethylamine (DIPEA) were sequentially added for 30 minutes. The reaction of the second step was performed by mixing at room temperature for 10 hours. Then, 13.37 g of N,N-diisopropylethylamine (DIPEA) and 25.60 g of 3-(triethoxysilyl) propyl isocyanate were added and then additionally mixed at 90° C. for 12 hours, thereby performing a third step. After lowering the temperature to room temperature, a base catalyst and a solvent were removed using a rotary evaporator, and then the reaction product was dried using a vacuum pump, thereby synthesizing a novel curing agent having an ester group and an amide group (curing agent equivalent: 148 g/Eq, [ester group]:[silylated amide group]=3.0:7.0 by mol).

### (4) Synthesis Example 4

The reaction was performed in the same manner as in Synthesis Example 3, except that 6.95 g of phenol and 9.55 g of N,N-diisopropylethylamine (DIPEA) were used in the reaction of the first step, 8.06 g of 3-aminophenol was used in the reaction of the second step, and 9.55 g of N, N-diisopropylethylamine (DIPEA) and 18.28 g of 3-(triethoxysilyl) propyl isocyanate were used in the reaction of the third step. A final compound obtained by the reaction was a novel curing agent (curing agent equivalent: 146 g/Eq, [ester group]:[silylated amide group]=5.0:5.0 by mol) having an ester group and an amide group.

### B. Preparation of Epoxy Cured Product and Evaluation of Physical Properties

### (1) Preparation and Evaluation of Dielectric Loss (D_{f}) and CTE Sample

### <Sample Preparation>

An epoxy resin, a curing agent, a thermoplastic resin, and silica were dissolved in methyl ethyl ketone according to the composition illustrated in Table 1 below. This mixture was stirred using a mixer at a speed of 1500 rpm for 30 minutes. Then, a curing catalyst was added thereto and the mixture was mixed to form a uniform solution and then cast onto a release film to achieve a uniform thickness, and the solvent of the casting film was dried in a convection oven preheated to 70° C. The dried film was cured under curing condition of 190° C. for 90 minutes to prepare a sample.

### <Dielectric Loss Evaluation>

The dielectric loss of the prepared epoxy film was evaluated using E5071C from Agilent Technologies at room temperature and 5.125 GHZ. A sample size for dielectric loss measurement was 60 mm×50 mm×0.1 mm.

### <CTE Evaluation>

The thermal expansion property of the epoxy cured product, i.e., the dimensional change with temperature, were evaluated using a thermo-mechanical analyzer. A sample size for CTE measurement was 4 mm×40 mm×0.1 mm.

### (2) Blister Evaluation:

An epoxy resin, a curing agent, a thermoplastic resin, and silica were dissolved in methyl ethyl ketone according to the composition in Table 1 below. The mixture was stirred using a mixer at a speed of 1500 rpm for 30 minutes. Then, the curing catalyst was added and the mixture was stirred to form a uniform solution. This solution was cast onto a release film with a uniform thickness and dried in a convection oven preheated to 70° C. to remove the solvent from the cast film. The dried film was laminated on a copper foil substrate using a film bonder (GMP Co., Ltd., GHQ-320AUTO), and then further cured in the oven. The curing conditions of the laminated film were 120° C. for 40 minutes and 180° C. for 20 minutes.

The cured epoxy film was immersed in a swelling solution Atotech Japan Co., Ltd., Swelling Dip Securiganth P) at 80° C. for 5 minutes. It was then immersed in a conditioning (or oxidizing) solution (Atotech Japan Co., Ltd., Concentrate Compact CP) at 80° C. for 20 minutes. Finally, it was immersed in a neutralizing solution (Atotech Japan Co., Ltd., Reduction Solution Securiganth P) at 50° C. for 5 minutes, and the remaining chemicals on a surface thereof were washed off to complete the desmear treatment.

The desmear-treated epoxy film was subjected to electroless copper plating and then heated at 150° C. for 1 hour. Then, electrolytic copper plating was performed, followed by heat treatment at 190° C. for 2 hours. The occurrence of blisters was observed and evaluated as follows.

### <Evaluation Criteria>

No blister occurrence: O

Blister occurrence: X

TABLE 1

| | | | Inventive | Inventive | Inventive | Inventive | Inventive | Comparative | Comparative |
|---|---|---|---|---|---|---|---|---|---|
| Epoxy Composition (g) | | | Ex. 1 | Ex. 2 | Ex. 3 | Ex. 4 | Ex. 5 | Ex. 1 | Ex. 2 |
| Epoxy Resin | Biphenyl Novolac Epoxy⁽¹⁾ | | 2.00 | | | 2.00 | | | |
| | Bisphenol A Novolac Epoxy⁽²⁾ | | | 2.00 | | | | 2.00 | |
| | Dicyclopentadiene Novolac | | | | 2.00 | | 2.00 | | 2.00 |
| | Epoxy⁽³⁾ | | | | | | | | |
| | Tetraglycidyl Ether of | | 2.00 | | | | | 2.00 | |
| | Methylenedianaline ⁽⁴⁾ | | | | | | | | |
| | 1,6-Naphthalene Epoxy⁽⁵⁾ | | | 2.00 | 2.00 | | | | |
| | Bisphenol A Epoxy⁽⁶⁾ | | | | | | 2.00 | | |
| | Biphenyl Epoxy₍₇₎ | | | | | 2.00 | | | 2.00 |
| Curing agent | Synthetic Ex. 1 | | 2.68 | | | | 0.29 | | |
| | Synthetic Ex. 2 | | | 0.96 | | | | | |
| | Synthetic Ex. 3 | | | | 1.27 | | | | |
| | Synthetic Ex. 4 | | | | | 0.67 | | | |
| | Active Ester Curing Agent⁽⁸⁾ | | | 2.05 | | 3.02 | 1.67 | | 3.98 |
| | Phenol Curing Agent⁽⁹⁾ | | | | 0.92 | | 0.81 | 2.65 | |
| Filler | Silica⁽¹⁰⁾ | | 19.42 | 20.36 | 18.00 | 22.33 | 19.66 | 19.34 | 23.17 |
| Thermoplastic | Polyvinly Butyral⁽¹¹⁾ | | 0.81 | 0.85 | 0.75 | 0.93 | 0.82 | 0.81 | 0.97 |
| Resin | | | | | | | | | |
| Curing Catalyst | 4-(dimethylamino)pyridine⁽¹²⁾ | | 0.02 | 0.02 | 0.02 | 0.02 | 0.02 | 0.02 | 0.02 |
| | Imidazole⁽¹³⁾ | | 0.04 | 0.04 | 0.04 | 0.04 | 0.04 | 0.04 | 0.04 |
| Evalutation | CTE | α₁ (T < T_{g}) | 18 | 18 | 17 | 19 | 17 | 19 | 20 |
| | | (ppm/° C.) | | | | | | | |
| | | α₂ (T > T_{g}) | 42 | 61 | 55 | 68 | 53 | 48 | 80 |
| | | (ppm/° C.) | | | | | | | |

| | | | | | | | |
|---|---|---|---|---|---|---|---|
| Dielectric Loss (D_{f}) | 0.0053 | 0.0038 | 0.0061 | 0.0035 | 0.0039 | 0.015 | 0.0039 |
| Blister Evaluation | 0 | 0 | 0 | 0 | 0 | Film was | X |
| | | | | | | peeled off: | |
| | | | | | | Unable to | |
| | | | | | | Evaluate | |

Note:
The compounds used in the above Table 1 are as follows:
⁽¹⁾Samhwa Paint Industry Co., Ltd., EEW 420 g/Eq, SSE-B20H
⁽²⁾Samhwa Paint Industry Co., Ltd., EEW 348 g/Eq, Serapoxy SHE0002
⁽³⁾Samhwa Paint Industry Co., Ltd., EEW 438 g/Eq, SSE-D30H
⁽⁴⁾ Sigma Aldrich, EEW 105 g/Eq
⁽⁵⁾DIC Co., Ltd., EEW 205 g/Eq, HP4770
⁽⁶⁾Sigma Aldrich, EEW 188.5 g/Eq, diglycidyl ether of bisphenol A
⁽⁷⁾Mitsubishi Chemical Co., Ltd., EEW 190, YX4000HK
⁽⁸⁾DIC Co., Ltd., 220 g/Eq, Epicolon HPC 8000L
⁽⁹⁾Meiwa Plastic Industries, 107 g/Eq, HF-1M
⁽¹⁰⁾Admatechs, SC2050-MTX
⁽¹¹⁾SEKISUI, BX-5
⁽¹²⁾Sigma aldrich
⁽¹³⁾Evonik, imidazole, CUREZOL ®

As may be seen from Table 1 above, the cured products of the epoxy compositions of Inventive Examples 1 to 5 including the novel curing agent of the present disclosure exhibited excellent (1) low dielectric loss and (2) desmearability. Specifically, the cured products of the epoxy compositions of Inventive Examples 1 to 5 comprising the novel curing agent of the present disclosure exhibited significantly lower dielectric loss values than the cured product using the conventional phenol curing agent (Comparative Example 1). Additionally, the films prepared with the compositions of Inventive Examples 1 to 5 had improved desmearability and did not cause blisters during high-temperature heat treatment. However, the sample of Comparative Example 1 comprising the phenol curing agent exhibited severe etching during the desmear treatment, and the epoxy film was removed from the substrate, making it impossible to manufacture a copper film sample.

Additionally, the epoxy cured product (Comparative Example 2) using the conventional an active ester curing agent exhibited an excellent dielectric loss value (i.e., low Df value), but the insufficient etching occurs during the desmear treatment, which leads to poor adhesion on copper foil. As a result, blisters were formed during high-temperature heat treatment.

From this, it may be concluded that the novel epoxy composition comprising the curing agent of the present disclosure exhibits excellent low dielectric loss characteristics and processability (specifically, desmearability) as compared to the epoxy composition comprising the conventional phenol curing agent alone (Comparative Example 1), and also improved exhibits desmearability as compared to the epoxy composition comprising the conventional ester-based curing agent alone (Comparative Example 2).

In this manner, the novel curing agent of the present disclosure improved the problems of not only the conventional phenol curing agent but also the ester curing agent.

Specifically, the conventional problems of the phenol curing agent and the ester curing agent could be improved not only by utilizing the novel curing agent of the present disclosure alone, but also by utilizing the novel curing agent of the present disclosure in combination with the phenol curing agent and/or the ester curing agent. Specifically, Example 3, in which the novel curing agent and the phenol curing agent were used together, exhibited significantly improved dielectric properties and desmearability as compared to the epoxy cured product (Comparative Example 1) in which the conventional phenol curing agent was used alone.

Additionally, in the case of Inventive Examples 2 and 4 comprising the novel curing agent of the present disclosure and the active ester curing agent, and Inventive Example 5 comprising the novel curing agent of the present disclosure, the active ester curing agent, and the phenol curing agent, the inventive examples not only exhibited excellent dielectric properties (i.e., low Df value) comparable to Comparative Example 2, but also exhibited excellent desmearability without blistering.

In addition to the dielectric loss and processability described above, thermal expansion characteristics are critical characteristics that determine the reliability and processability of epoxy components. In this regard, in the case of Inventive Examples 1 to 5 comprising the novel curing agent of the present disclosure, excellent dielectric characteristics (i.e., low Df value) comparable to those of Comparative Example 2 comprising the conventional active ester curing agent were exhibited, and no blistering occurred, and improved thermal expansion characteristics (i.e., low CTE1 and CTE2 values) were exhibited as compared to Comparative Example 2.

What is claimed is:

1. A curing agent represented by formula (1): 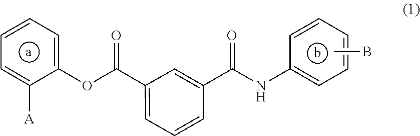
in which:
A is hydrogen or an allyl group, and
B is an OH group or a 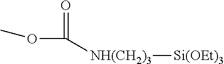
(where Et is an ethyl group) group.

2. A method for preparing a curing agent in which B is an OH group in formula (1), the method comprising:
a first step of the formation of an ester group by mixing isophthaloyl chloride with at least one of phenol and 2-allyl phenol; and
a second step of the formation of an amide functional group by mixing a reaction product of the first step with at least one of m-amino phenol, p-amino phenol and o-amino phenol, 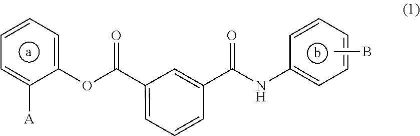
in which:
A is hydrogen or an allyl group, and
B is an OH group.

3. A method for preparing a curing agent in which B is a 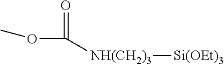
(where Et is an ethyl group) group in formula (1), the method comprising:
a first step of the formation of an ester group by mixing isophthaloyl chloride with at least one of phenol and 2-allyl phenol;
a second step of the formation of an amide functional group by mixing a reaction product of the first step with at least one of m-amino phenol, p-amino phenol and o-amino phenol; and
a third step of the formation of an alkoxysilyl group by mixing a reaction product of the second step with 3-(triethoxysilyl) propyl isocyanate, 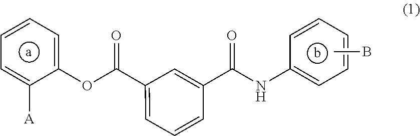
in which:
A is hydrogen or an allyl group, and B is a 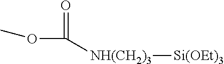
(where Et is an ethyl group) group.

4. The method according to claim 2,
wherein in the first step, at least one of phenol and 2-allyl phenol is mixed in an amount of 0.6 equivalents to 1.4 equivalents with respect to 1 equivalent of isophthaloyl chloride, and
in the second step, the reaction product of the first step is mixed with at least one of m-amino phenol, p-amino phenol and o-amino phenol in an amount of 0.6 equivalents to 1.4 equivalents with respect to one 1 equivalent of isophthaloyl chloride used in the first step.

5. The method according to claim 3,
wherein in the first step, at least one of phenol and 2-allyl phenol is mixed in an amount of 0.6 equivalents to 1.4 equivalents with respect to 1 equivalent of isophthaloyl chloride, and
in the second step, the reaction product of the first step is mixed with at least one of m-amino phenol, p-amino phenol and o-amino phenol in an amount of 0.6 equivalents to 1.4 equivalents with respect to one 1 equivalent of isophthaloyl chloride used in the first step.

6. The method according to claim 3,
wherein in the third step, 0.1 to 1.0 equivalents of 3-(triethoxysilyl) propyl isocyanate are mixed with respect to 1 equivalent of the hydroxyl group of the reaction product of the second step.

7. An epoxy composition, comprising:
an (A) epoxy resin and a (B) curing agent,
wherein as the (B) curing agent, (i) the curing agent represented by formula (1) of claim 1 is used alone, or (ii) the curing agent (a) represented by formula (1) of claim 1 and at least one curing agent (b) of an active ester-based curing agent and a phenol-based curing agent are used together.

8. The epoxy composition according to claim 7,
wherein when the curing agent (a) represented by formula (1) of claim 1 and at least one curing agent (b) of the active ester-based curing agent and the phenol-based curing agent are used together, the curing agent (a) and the curing agent (b) are used in a mole ratio of 0.5:9.5 to 9.5:0.5.

9. A cured product of the epoxy composition according to claim 7.

10. An article comprising the epoxy composition according to claim 7.

11. The article according to claim 10,
wherein the article is an insulating film, an epoxy film for semiconductor packaging, an adhesive film, a build-up film, a substrate film, or an epoxy molding material.
